(19) United States
(12) Patent Application Publication
(10) Pub. No.: US 2024/0221870 A1
(43) Pub. Date: Jul. 4, 2024

(54) SYSTEM AND METHOD FOR TRANSLATING IMAGE OF STRUCTURAL FORMULA OF CHEMICAL MOLECULE INTO TEXTUAL IDENTIFIER THEREFOR

(71) Applicant: Quantiphi Inc, Marlborough, MA (US)

(72) Inventors: Dagnachew Birru, Marlborough, MA (US); Sofia P. Moschou, Reading (GB); Mahdieh Khalilinezhad, Toronto (CA)

(73) Assignee: Quantiphi Inc, Marlborough, MA (US)

(21) Appl. No.: 18/147,052

(22) Filed: Dec. 28, 2022

(51) Int. Cl. G16C 20/20; G06F 40/284; G06N 3/0442; G06N 3/0455; G16C 20/70; G16C 20/80

(52) U.S. Cl. CPC G16C 20/20; G06F 40/284; G06N 3/0442; G06N 3/0455; G16C 20/70; G16C 20/80

(57) ABSTRACT

Disclosed is a system and a method for translating an image of a structural formula of a chemical molecule into a textual identifier therefor utilizing unique tokens for each of known entities. The method comprises pre-processing the image of the structural formula to generate a standardized image; processing the standardized image using an encoder-decoder architecture, wherein an encoder generates embeddings for features in the standardized image and a decoder is implemented to associate each of the features to one of the unique tokens; recurrently processing each of the features in the standardized image for predicting corresponding unique token to generate multiple possible sequences, and dynamically calculating a correctness probability for each of the generated sequences; selecting one of the sequences with highest calculated correctness probability; and generating the textual identifier, as an output, based on the selected one of the sequences.

### TECHNICAL FIELD

The present disclosure generally relates to data translation models. Specifically, the present disclosure relates to systems and methods translating an image of a structural formula of a chemical molecule into a textual identifier therefor.

### BACKGROUND

Traditionally, organic chemists tend to draw out molecular work with their skeletal formulas i.e., a structural notation used for centuries. Further, publications have been annotated with machine-readable chemical descriptions such as, the International Chemical Identifier (InChI), a textual identifier for chemical substances, designed to provide a standard way to encode molecular information and to facilitate the search for such information in databases and on the web. Particularly, representation and interpretation (or translation) of a molecular entity, uniquely encoded, plays a vital role in utilizing the vast amount of existing data for driving innovations across the chemical industry. Tools to curate chemistry literature would be a significant benefit to researchers. If successful, it may help chemists expand access to collective chemical research. In turn, this would speed up research and development efforts in many key fields by avoiding repetition of previously published chemistries and identifying novel trends via mining large data sets.

Automated recognition of optical chemical structures, with the help of machine learning, could speed up research and development efforts. Although, there exists some methods for performing such recognition, interpretation and/or translation of molecular entities; however, implementing such a method for millions of scanned documents spanning decades is unfeasible and further for enabling automatic search for specific chemical depictions is currently not possible since most public data sets are too small and/or without enough quality variability to support modern machine learning models. Further, such existing solutions (or tools) provide a maximum of 90% accuracy and that too under optimal conditions.

Moreover, historical sources often have some level of image corruption, that reduces performance of existing ML techniques. In these cases, such solutions are highly time-consuming and require a lot of manual work to reliably convert scanned chemical structure images into a machine-readable format.

Therefore, in light of the foregoing discussion, there exists a need to overcome the aforementioned drawbacks associated with the existing solutions and provide an improved system and method for translating an image of a structural formula of a chemical molecule into a textual identifier therefor.

### SUMMARY

The present disclosure seeks to provide a method for translating an image of a structural formula of a chemical molecule into a textual identifier therefor. The present disclosure also seeks to provide a system for translating an image of a structural formula of a chemical molecule into a textual identifier therefor. An aim of the present disclosure is to provide a solution that overcomes at least partially the problems encountered in prior art.

In one aspect, an embodiment of the present disclosure provides a system for translating an image of a structural formula of a chemical molecule into a textual identifier therefor, the system comprising:
a database configured to store unique tokens defined for each of known entities in chemical molecules; and
a processing arrangement configured to:
   pre-process the image of the structural formula of the chemical molecule to generate a standardized image of the structural formula based on predefined parameters;
   process the standardized image of the structural formula using an encoder-decoder architecture, wherein an encoder is implemented to generate embeddings for features in the standardized image of the structural formula and a decoder is implemented to utilize the generated embeddings along with an attention mechanism to associate each of the features in the standardized image of the structural formula to one of the unique tokens;
   recurrently process each of the features in the standardized image of the structural formula for predicting corresponding unique token based on the associated unique tokens therewith to generate multiple possible sequences complementary to the textual identifier, and dynamically calculate a correctness probability for each of the generated multiple possible sequences based on a confidence of each prediction of corresponding unique tokens involved therein;
   select one of the multiple possible sequences with highest calculated correctness probability; and
   generate the textual identifier, as an output, for the image of the structural formula of the chemical molecule based on the selected one of the multiple possible sequences.

In another aspect, an embodiment of the present disclosure provides a computer readable storage medium having computer executable instruction that when executed by a computer system, causes the computer system to execute a method for translating an image of a structural formula of a chemical molecule into a textual identifier therefor utilizing unique tokens generated for each of known entities in chemical molecules, the method comprising:
pre-processing the image of the structural formula of the chemical molecule to generate a standardized image of the structural formula based on predefined parameters;
processing the standardized image of the structural formula using an encoder-decoder architecture, wherein an encoder is implemented to generate embeddings for features in the standardized image of the structural formula and a decoder is implemented to utilize the generated embeddings along with an attention mechanism to associate each of the features in the standardized image of the structural formula to one of the unique tokens;
recurrently processing each of the features in the standardized image of the structural formula for predicting corresponding unique token based on the associated unique tokens therewith to generate multiple possible sequences complementary to the textual identifier, and dynamically calculating a correctness probability for each of the generated multiple possible sequences based on a confidence of each prediction of corresponding unique tokens involved therein;
selecting one of the multiple possible sequences with highest calculated correctness probability; and
generating the textual identifier, as an output, for the image of the structural formula of the chemical molecule based on the selected one of the multiple possible sequences.

Embodiments of the present disclosure substantially eliminate or at least partially address the aforementioned problems in the prior art, and enables translation of chemical images in an accurate and efficient manner.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 illustrates a block diagram of a system for translating an image of a structural formula of a chemical molecule into a textual identifier therefor, in accordance with an embodiment of the present disclosure;

FIG. 2 illustrates a flowchart listing steps involved in a method for translating an image of a structural formula of a chemical molecule into a textual identifier therefor, in accordance with an embodiment of the present disclosure;

FIGS. 3A and 3B are schematic block diagrams depicting pipelines involved in the method of FIG. 2 for translating an image of a structural formula of a chemical molecule into a textual identifier therefor, during a training phase and an inference phase, in accordance with an embodiment of the present disclosure;

FIG. 4 is an exemplary flowchart of steps for translating an exemplary image into a textual description therefor as involved in the method of FIG. 2, in accordance with an embodiment of the present disclosure

FIG. 5 is a simplified pipeline of an embedding generation process implemented in the method of FIG. 2 for translating an image of a structural formula of a chemical molecule into a feature vector to be utilized for generating the textual identifier; and

FIGS. 6A to 6F are exemplary depictions of the generation of multiple possible sequences complementary to the textual identifier via the decoder of the processing arrangement, in accordance with one or more embodiments of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

In one aspect, an embodiment of the present disclosure provides a system for translating an image of a structural formula of a chemical molecule into a textual identifier therefor, the system comprising:
a database configured to store unique tokens defined for each of known entities in chemical molecules; and
a processing arrangement configured to:
   pre-process the image of the structural formula of the chemical molecule to generate a standardized image of the structural formula based on predefined parameters;
   process the standardized image of the structural formula using an encoder-decoder architecture, wherein an encoder is implemented to generate embeddings for features in the standardized image of the structural formula and a decoder is implemented to utilize the generated embeddings along with an attention mechanism to associate each of the features in the standardized image of the structural formula to one of the unique tokens;
   recurrently process each of the features in the standardized image of the structural formula for predicting corresponding unique token based on the associated unique tokens therewith to generate multiple possible sequences complementary to the textual identifier, and dynamically calculate a correctness probability for each of the generated multiple possible sequences based on a confidence of each prediction of corresponding unique tokens involved therein;
   select one of the multiple possible sequences with highest calculated correctness probability; and
   generate the textual identifier, as an output, for the image of the structural formula of the chemical molecule based on the selected one of the multiple possible sequences.

In another aspect, an embodiment of the present disclosure provides a computer readable storage medium having computer executable instruction that when executed by a computer system, causes the computer system to execute a method for translating an image of a structural formula of a chemical molecule into a textual identifier therefor utilizing unique tokens generated for each of known entities in chemical molecules, the method comprising:
pre-processing the image of the structural formula of the chemical molecule to generate a standardized image of the structural formula based on predefined parameters;
processing the standardized image of the structural formula using an encoder-decoder architecture, wherein an encoder is implemented to generate embeddings for features in the standardized image of the structural formula and a decoder is implemented to utilize the generated embeddings along with an attention mechanism to associate each of the features in the standardized image of the structural formula to one of the unique tokens;
recurrently processing each of the features in the standardized image of the structural formula for predicting corresponding unique token based on the associated unique tokens therewith to generate multiple possible sequences complementary to the textual identifier, and dynamically calculating a correctness probability for each of the generated multiple possible sequences based on a confidence of each prediction of corresponding unique tokens involved therein;
selecting one of the multiple possible sequences with highest calculated correctness probability; and
generating the textual identifier, as an output, for the image of the structural formula of the chemical molecule based on the selected one of the multiple possible sequences.

Throughout the present disclosure, the term “textual identifier” refers to a machine-readable symbol or text configured to represent given element(s) of the chemical molecule (or compound) i.e., the structural formula including elements and chemical bonds formed therebetween. The textual identifier may be at least one of a keyword or a sentence, a symbol, or an international chemical identifier (InChI), or a simplified molecular-input line-entry (SMILE) textual identifier, and the like. The term “structural formula” refers to a visual or graphical representation of molecular structure of the chemical molecule, depicting the arrangement of elements (or atoms) therein in a two-dimensional (2D) or three-dimensional (3-D) space, wherein chemical bonding within the molecular structure may also be depicted, either explicitly or implicitly. In an exemplary scenario, wherein an image of a structural formula of a chemical molecule of caffeine is translated into an InChI textual identifier represented by: 1S/C8H10N4O2/c1-10-4-9-6-5(10)7(13)12(3)8(14)11(6)2/h4H.1-3H3.

The present disclosure provides a system for translating an image of a structural formula of a chemical molecule into a textual identifier therefor. Alternatively stated, the present system is configured to translate the image of a structural formula, associated with a chemical molecule, into the textual identifier. The system of the present disclosure is configured for molecular translation of any image of a chemical molecule into the textual identifier for enabling interpretation, analysis and processing thereof, i.e., translating input images into textual form for allowing user(s) to utilize and/or process the translated textual identifiers such as, for performing research and/or experiments. Beneficially, the system of the present disclosure enables interpretation of old (or existing) chemical images (or data) into a machine-readable (or computer readable) form such that the existing data may be processed to allow a user (such as, scientists, chemists, or researchers) to expand their access to collective scientific or chemical research and improve the efficiency of research and development efforts. Moreover, such an implementation of the system enables identification of novel trends via mining and/or analysis of large public or private datasets. Moreover, the present system also enables user(s) to avoid repetition of existing research and/or publication owing to the simple, faster and efficient processing of data via the system. It will be appreciated that although the present system is directed towards translation of images of structural formula of chemical molecules; however, any other type of image or video may also be interpreted or processed via the present system without any limitations.

The present system comprises a database configured to store unique tokens defined for each of known entities in chemical molecules. The database is configured to store unique tokens defined for each of the known entities in the chemical molecule whose image is being translated for enabling further processing and/or analysis of the image of the structural formula of the chemical molecule via the stored unique tokens. The term “unique token” refers to words, sub-words, symbols or individual characters associated with each of the known entities present in the input image i.e., the image of the structural formula of the chemical molecule. In an example, wherein the chemical molecule is CO2, the known entities are carbon ‘C’, oxygen ‘O’ and two double bonds ‘═’ therebetween, the database is configured to store the defined unique tokens associated with each of the elements in the structural formula of the chemical molecule whose image is being processed via the system. Alternatively stated, the system may be configured to process the structural formula of the chemical molecule to be represented by the constituent parts i.e., the individually defined unique tokens of the input image for allowing further processing and/or analysis thereof in an efficient manner. It will be appreciated by a person skilled in the art that the database is a unique chemical identifier database comprising an extensive list of all possible textual chemical identifiers, wherein the tokenization of each of the known entities in the chemical molecule is performed during training based on the textual chemical identifier in the database.

The system further comprises a processing arrangement. The term “processing arrangement” as used herein refers to a structure and/or module that includes programmable and/or non-programmable components configured to store, process and/or share information and/or signals relating to the method for translating an image of a structural formula of a chemical molecule into a textual identifier therefor. The processing arrangement may be a controller having elements, such as a display, control buttons or joysticks, processors, memory and the like. Typically, the processing arrangement is operable to perform one or more operations for translating the image of a structural formula of a chemical molecule into a textual identifier therefor. In the present examples, the processing arrangement may include components such as memory, a processor, a network adapter and the like, to store, process and/or share information with other computing components, such as, a user interface, a user device, a remote server unit, a database arrangement. Optionally, the processing arrangement includes any arrangement of physical or virtual computational entities capable of enhancing information to perform various computational tasks. Further, it will be appreciated that the processing arrangement may be implemented as a hardware processor and/or plurality of hardware processors operating in a parallel or in a distributed architecture. Optionally, the processing arrangement is supplemented with additional computation system including neural networks such as, Artificial (ANN), Convolutional (CNN), Recurrent (RNN), RCNNs, Multilayer Perceptron (MLP) and so forth and hierarchical clusters of pseudo-analog variable state machines implementing artificial intelligence algorithms. Optionally, the processing arrangement is implemented as a computer program that provides various services (such as database service) to other devices, modules or apparatus. Optionally, the processing arrangement includes, but is not limited to, a Tensor Processing Unit (TPU), Graphical Processing unit (GPU), a microprocessor, a micro-controller, a complex instruction set computing (CISC) microprocessor, a reduced instruction set (RISC) microprocessor, a very long instruction word (VLIW) microprocessor, Field Programmable Gate Array (FPGA) or any other type of processing circuit, for example as aforementioned. Additionally, the processing arrangement may be arranged in various architectures for responding to and processing the instructions for generating the notations via the method.

Herein, the system elements may communicate with each other using a communication interface. The communication interface includes a medium (e.g., a communication channel) through which the system components communicate with each other. Examples of the communication interface include, but are not limited to, a communication channel in a computer cluster, a Local Area Communication channel (LAN), a cellular communication channel, a wireless sensor communication channel (WSN), a cloud communication channel, a Metropolitan Area Communication channel (MAN), and/or the Internet. Optionally, the communication interface comprises one or more of a wired connection, a wireless network, cellular networks such as 2G, 3G, 4G, 5G mobile networks, and a Zigbee connection.

The processing arrangement is configured to pre-process the image of the structural formula of the chemical molecule to generate a standardized image of the structural formula based on predefined parameters. Herein, the processing arrangement is configured to pre-process the image of the structural formula of the chemical molecule to generate the standardized image of the structural formula based on the predefined parameters for enabling further processing thereof in a quick and efficient manner. Typically, the processing arrangement is configured to pre-process the image of the structural formula of the chemical molecule (also interchangeably referred to as, ‘input image’ or ‘image of chemical molecule’ or ‘chemical image’ in the present disclosure) to convert and compress the input image into a machine-readable form i.e., the standardized image, based on the predefined parameters. The processing arrangement is configured to successfully capture spatial and temporal dependencies in the input chemical image via application of relevant filters based on the one or more predefined parameters. Such a pre-processing enables encoder-decoder architecture to perform better or accurate fitting to the input image due to the compression of multiple input images into a singular standardized image and further improve the computational efficiency of the processing arrangement due to the reduced number of input-output (I/O) operations. Alternatively stated, the system or the processing arrangement may be trained to understand the sophistication of the input image of the structural formula of the chemical molecule in an effective and accurate manner for allowing further processing thereof. In some embodiments, the preprocessing of the image of the structural formula of the chemical molecule comprises performance of one or more processing techniques including, but not limited to, sampling the one or more unique tokens, convolutions, sub-sampling, augmentation, cleaning, rescaling, on the one or more unique tokens associated with the input image of the chemical molecule to generate the standardized image i.e., a light-weight form of the input image to further enable efficient processing via the system.

Throughout the present disclosure, the term “standardized image” refers to a machine-readable molecular representation of the image of the structural formula of the chemical image received as input for further processing via the system. For example, the standardized image may be represented by an array of numbers or strings (or texts), a set of feature vectors, graphical representations, computer records (such as, TensorFlow records records), and the like. The processing arrangement may be configured to generate the standardized image via feeding through a normalization layer for scaling of the input chemical image and/or the one or more parameters associated therewith via standard statistical techniques such as, Z-score normalization, Notably, the standardized image is generated to uniformize or standardize the input image based on the one or more parameters and/or features of the image of the structural formula of the chemical molecule. The standardized image (often known as, “descriptor” of feature vectors) encodes chemical identity of the chemical molecule of the input image in terms of its chemical composition and atomic configuration, wherein the conversion of the input image of the structural formula of the chemical molecule to the standardized image is required to enable the system to efficiently process a large number of chemical images and associated structures. It will be appreciated that the processing arrangement may further use distance measures such as K-nearest neighbour (KNN), K-means clustering, principal component analysis (PCA), and the like, during or after generation of the standardized image without any limitations

In an embodiment, the processing arrangement may be configured to pre-process the input image by conversion or compression into TensorFlow (TF) records. The TF records may be configured to cluster the input image of the chemical molecule with one or more annotations or texts for enabling classification and processing thereafter. Herein, the pre-processing of input data via the processing arrangement involves image analysis and language analysis (e.g., chemical notations) performed parallelly i.e., in separate processers of the processing arrangement to improve the speed of the pre-processing step and thereby improve the efficiency of the system by enabling quicker output provision to an encoder for further processing or training via the system. In another embodiment, the standardized image may be a latent space representation. The processing arrangement may be configured to received raw pixels of the input chemical image and encode a final layer with high-level features extracted therefrom into the latent space representation or the TF record for enabling the model to perform tasks (e.g., classification) efficiently via parallel processing of the light-weight or compressed standardized image via the processing arrangement. Notably, the standardized image may be generated to counter inherent variances for computing accurate outputs, for example, rotational invariance, translational invariance, and permutational invariance during generation of the standardized image.

The “predefined parameters” refer to customized processing parameters configured to enable the system to further analyze or process input data i.e., the image of the structural formula of the chemical molecule to generate the standardized data. The one or more predefined parameters may be at least one of a pixel parameter, a color parameter, a crop parameter, a size parameter, and the like, associated with the image of the structural formula of the chemical molecule. The pixel parameter refers to pixel values of the input image such as, RGB values, resolution, etc. The size parameter refers to height and width of the input image. For example, aspect ratio. The color parameter refers to color attributes associated each or some of the pixel(s) in the input image. The processing arrangement may be configured to automatically and/or recursively adjust the parameters for future implementations upon training therefor. It will be appreciated that any other predefined parameter may be utilized via the system to reduce the memory requirement for improved efficiency of the system without any limitations.

In some embodiments, the preprocessing of the image of the structural formula of the chemical molecule, via the processing arrangement, may be performed by extracting one or more features from the input chemical image and thereby associating the features to each of the one or more unique tokens to generate the standardized image, wherein the features may be extracted using convolution feature extraction on each of the one or more unique tokens. The processing arrangement may be further configured to utilize the extracted features associated with the unique tokens for representing the input chemical image to a lower dimensionality space (i.e., a feature map) associated with the one or more features using feature extraction. In an exemplary scenario, wherein an input chemical image is one of caffeine, the processing arrangement may generate a 196×1 feature vector to be further processed for generating the corresponding textual chemical identifier. Notably, the decoder (for example, using an attention-based LSTM model) utilized via the system is configured to automatically learn description or analysis of content and context of the input chemical images. The processing arrangement may be trained in a deterministic manner using standard backpropagation techniques. Notably, the attention mechanism utilized via the processing arrangement improves the performance of accurate textural identifier (or textual labels) prediction on the chemical images. Additionally, the generated feature vector and may be utilized by the model to be trained to automatically locate corresponding features in the feature vector representation of the input chemical and processing salient objects (or unique tokens) while generating the corresponding textual identifier on the input chemical images and/or datasets such as, datasets pre-stored in the database, or benchmark datasets such as, but not limited to, Flickr30k and MS COCO, or other opensource datasets.

The processing arrangement is further configured to process the standardized image of the structural formula using an encoder-decoder architecture. Throughout the present disclosure, the term “encoder-decoder architecture” refers to a structure and/or module comprising a set of encoders and decoders operatively coupled with each other and configured to enable the system (or processing arrangement) to generate the textual identifier accurately describing the image of the structural formula of the chemical molecule. Typically, the encoder-decoder architecture is a part of the processing arrangement and may be configured to receive the standardized image of the chemical molecule for further processing to output a sequence of words i.e., the textual identifier, wherein the encoder-decoder architecture may comprise a suite of encoders and decoders to enable parallel processing for generation of the textual identifier in a quick and efficient manner.

In one or more embodiments, the processing arrangement implements a tensor workflow for the encoder-decoder architecture for processing the standardized image of the structural formula as a TensorFlow TFRecord. The TF records may be configured to cluster (or annotate) the standardized image of the chemical molecule with one or more annotations or classification for enabling efficient processing thereafter via the processing arrangement. Herein, the pre-processing of input data via the processing arrangement involves image analysis and language analysis (e.g., chemical notations) performed parallelly i.e., in separate processers of the processing arrangement to improve the speed of the pre-processing step and thereby improve the efficiency of the system by enabling quicker output provision to a decoder for further processing or training via the system.

Herein, the encoder of the encoder-decoder architecture is implemented to generate embeddings for associating features to each of the unique tokens in the standardized image of the structural formula of the chemical molecule. The “encoder” refers to a structure and/or module configured to perform computer vision tasks convert the standardized image into a required format that can be processed via the decoder of encoder-decoder architecture. Herein, the encoder is configured to generate embeddings for features in the standardized image and thereby enable the system to translate the image of the chemical molecule into the textual identifier. The encoder may be built by stacking a set of convolutions neural networks (CNN)) configured for parallel encoding of the input standardized image for enabling further processing via the decoder. In an example, the encoder may be operable to convert a drawing (image) into one or more words (or texts), vectors or embeddings. The term “embedding” as used herein refers to encapsulated data representing the extracted features in the standardized image for description thereof to enable further analysis and/or processing via the system. For example, the embeddings may be token embeddings or image embeddings representing dense vector representations of the image of the chemical molecule, or a text embedding associated therewith, to be decoded via the decoder for classification and/or processing. In the context of molecular translation of chemical images via the system, the embeddings are generated for features in the standardized image and may be an embedding vector representing features of each of the one or more unique tokens associated with the input image of the chemical molecule in a reduced or compressed format to enable faster processing via the decoder of the encoder-decoder architecture. For example, the description of the standardized image can be vectorized into a sparse one-dimensional or two-dimensional matrix based on the needs of the implementation for feeding into a decoder or classifier of the encoder-decoder architecture. Herein, the encoder may map each of features in the standardized image based on the one or more parameters to generated the contextualized embeddings, which can further act as input for various downstream processing tasks via the system. Additionally, positional embeddings may be added to the generated embeddings to retain positional information of each of the one or more features in the standardized image. For example, via 1-D positional embeddings, or 2-D aware positional embeddings, wherein the resulting sequence of embedding vectors serves as input to the encoder.

In one or more embodiments, the encoder implements one or more of: an EfficientNet encoder, an EfficientNetV2 encoder, a Vision Transformer (ViT) encoder. In some examples, the encoder implements the EfficientNetB #(0, 4, 5, 7) encoder. In another embodiment, the encoder implements the EfficientNetV2- #(B0, B3, M, L). In such embodiments, the standardized image received as input to the encoder is encoded to generate convolutional layers for each unique token in the standardized image. Typically, the generated convolutional layers are processed via the activation function, wherein the activation function may be selected from at least one of a binary step function, a piece-wise linear activation function or non-linear activation function, a sigmoid function, a hyperbolic tangent (tanh), a rectified linear unit (ReLU), a parametric ReLU, an exponential linear unit (ELU), a swish function, or a scaled ELU (SELU) activation function, to process the standardized image. It will be appreciated that the encoder may utilize the aforementioned functions based on the type of prediction and one or more activation functions may be simultaneously utilized via the processing arrangement without limitations.

In one or more embodiments, the encoder may be configured to generate the one or more embeddings by performing one or more processing techniques to filter the standardized image for detecting the input chemical image and extracting specific features (or parameters) therein via implementation of convolutional layers for enabling detection and extraction of required features of each of the unique tokens in the filtered standardized image. Further, the encoder may be configured to convert the extracted features into a feature map that may be passed through the selected activation function, wherein weights (or kernels) may be assigned via the system to each unique token (or parameter) of the standardized image to define the interconnection between the convolutional layers and thereby extract the features from the standardized image. Further, the encoder may compress or condense the standardized image to enhance and/or improve the extracted features of the standardized image through the generated pooling layers for down sampling to be further processed via the decoder. Furthermore, the encoder may be configured to convert the intermediate feature maps into the embeddings i.e., one or more flattened layers (or feature vectors) that are suitable for efficient processing via the decoder of the encoder-decoder architecture of the processing arrangement and thereby enabling generation of accurate textual identifiers associated with the input image of the chemical molecule in an efficient manner.

In yet another embodiment, the encoder implements a customized vision transformer. The customized vision transformer (VIT) refers to an attention-based transformer encoder utilized to receive preprocessed image i.e., the standardized image, divide or break the standardized image into multiple smaller patches/chunks/pieces, add positional embeddings to each of the smaller chunks and store context associated with the location or positional embeddings of each of the multiple patches with respect to the original image, and then processes each patch in the customized ViT to generate a flattened feature vector similar to any other encoder, ready to be processed via the decoder. Beneficially, such a break down via the customized ViT encoder enables processing of the original large image into smaller parts to improve the speed of the encoding processes owing to simultaneous parallel processing of each patch via the VIT encoder.

Further, the encoder implements the customized vision transformer to determine relationships between visual semantic concepts in the standardized image i.e., for enabling interpretation of the standardized image and the one or more unique tokens associated therewith using the associated features. The encoder implementing the customized ViT transformer may be configured to dynamically extract a set of features from the standardized image to obtain a compressed representation of the image of the structural formula of the chemical molecule. Further, the encoder is configured to implement the customized ViT transformer to obtain features of the standardized image required to generate densely modelled semantic relationships therebetween and enabling interpretation via the system. Beneficially, such an implementation of token-based representation of the image of the chemical molecule enables customized vision transformer to improve the efficiency and processing speed of the processing arrangement such as, during feature extraction of the standardized image. Typically, the visual transformers may be configured to replace the last stage of convolutions performed via the encoder based on the generated embeddings reduces the computational time and at the same time improves the accuracy of the output image sequences and thereby transmit the generated embeddings to the decoder (such as, an MLP head) for further processing.

In one or more embodiments, the encoder implementing the customized vision transformer may be configured to receive an input standardized image, for which the encoder is configured to reshape the standardized image into a feature vector based on the features or parameters of the standardized image such as, based on the resolution of the original image, number of channels, resolution of features, and total number of features, allowing the encoder to generate effective input sequence lengths for the decoder of the encoder-decoder architecture. The decoder may be a transformer configured to utilize constant latent vector (or embedding) sizes, for example, ‘x’ dimensions, through each layer, such that the encoder may generate the embeddings by flattening the standardized image and mapping thereof to the latent vector size i.e., X dimensions with a trainable linear projection. Notably, the generated embeddings are dynamically trained using sequences of pooling layers (configured for down sampling) of the filtered standardized image generated for efficient processing via the processing arrangement, wherein a state at the output of the encoder serves as an image representation that may be processed via decoders of the processing arrangement during pre-training and/or fine-tuning.

In one or more embodiments, the encoder, in the processing arrangement, is configured to implement a mixed precision accuracy scheme to the embeddings for the features in the TensorFlow TFRecord. Herein, based on the requirements of the implementation i.e., whether an improved computational speed for processing large input image datasets is required i.e., for faster generation of embeddings, or whether an improved accuracy for enabling updation of generated embeddings for the features, the encoder implements the mixed accuracy scheme to increase the computational speed of the encoder while maintaining a high accuracy during the generation of the embeddings in a quick and efficient manner i.e., complimented with the faster processibility of the utilized TFRecords. The “mixed precision accuracy scheme” relates to the mechanism by which the processing arrangement and/or the encoder of the encoder-decoder architecture is configured to improve the rate of generation of the embeddings for features in the TFRecord. The mixed precision accuracy scheme implemented via the encoder involves usage of two data types i.e., a 16-bit and a 32-bit floating-point type to be implemented via the model during training or inference thereof to make the encoding process faster while occupying lesser space to improve the efficiency of the system. The mixed precision accuracy scheme is applied throughout the entire algorithm i.e., from the preprocessing to the final prediction of the textual identifier to beneficially optimize the entire encoder-decoder architecture. Moreover, the mixed precision accuracy scheme enables performance of a majority of computations of the processing arrangement, apart from the output (or minority operations) with 16-bit accuracy, wherein all the variables are kept at a 32-bit i.e., higher accuracy and thus, using the higher 32-bit accuracy for processing outputs, variables and important computations, while the remainder (or majority operations) are performed with 16-bit accuracy.

Typically, the mixed precision accuracy scheme may involve configuring the encoder to process a part of the standardized image (or input data) using 32-bit floating point data types for numeric stability and 16-bit floating point data types for faster processing of the remaining part of the standardized image (or any other input data) to improve performance of the processing arrangement by up to 300% in comparison to modern GPUs and approximately 60% better performance in comparison to TPUs. It will be appreciated that the encoders may be selected based on the data type utilized, for example, the processing arrangement may select one or more GPUs to run operations in float16 rather than float32, and one or more TPUs to run operations in float16 faster than float32.

Further, in the processing arrangement, a decoder is implemented to utilize the generated embeddings along with an attention mechanism to associate each of the features in the standardized image of the structural formula to one of the unique tokens. The term “decoder” refers to a structure and/or module configured to decode or predict an output at a particular time instant based on the input received i.e., the embeddings (or encoder vectors) from the encoder that acts as an initial hidden state of the decoder in the encoder-decoder architecture. Further, the decoder may act as a classification head implementing a multilayer perceptron (MLP) head having at least one hidden layer generated during the pre-processing step to be converted to a single linear layer or embedding to be further utilized for generation of accurate textual identifiers for the image of the structural formula of the chemical molecule via the processing arrangement or system. Herein, the decoder is configured to utilize the generated embeddings (or encoded vectors) for processing thereof using the attention mechanism to associated each of the features in the standardized image of the structural formula of the chemical molecule to one of the unique tokens. Notably, the “attention mechanism” refers to a mechanism or process of interpretation and extraction of contextual information from the generated embeddings for association with each of the features in the standardized image of the structural formula of the chemical molecule. In one or more embodiments, the decoder implements one or more attention mechanisms including, but not limited to, Bahdanau Attention, Transformer self-attention, for associating each of the features in the standardized image to the at least one of the unique tokens. In one or more embodiments, the decoder implements one or more of: a Recurrent Neural Network (RNN) with a Gated Recurrent Unit (GRU) decoder, a RNN with a Long Short-Term Memory (LSTM) decoder, a Transformer with self-attention decoder to associate the features of the standardized image to one of the unique tokens. Beneficially, the implementation of the attention mechanism to decode the generated embeddings improves the accuracy of the output and therefor the classification of the input standardized image and/or the unique tokens associated therewith in an efficient manner.

In some embodiments, the decoder is a Transformer and comprise inherent benefits of increased computational efficiency and accuracy in comparison to RNNs; since, the transformer decoder is enabled to parallelly process the target textual identifier i.e., the InChI text during training and obtain information from previous and future states via the combination of the look ahead masking operation and the attention mechanisms implemented via the system.

Notably, during training, some decoders of the encoder-decoder architecture, such as, a RNN decoder with a gated recurrent unit (GRU), or a RNN decoder with LSTMs, are configured for recurrent processing for improved efficacy, while, some decoders of the encoder-decoder architecture such as, transformer decoders, are configured for parallel processing (or decoding) for improved efficiency. Moreover, during inference, each of the decoders in the encoder-decoder architecture are configured for recurrent prediction i.e., token-by-token, until the entire possible sequences are generated.

In one or more embodiments, the decoder implements an accelerated linear algebra (XLA) scheme to associated the features of the standardized image to one of the unique tokens. Herein, the decoder may be a domain-specific compiler for linear algebra configured to accelerate the machine learning (ML) models or libraries utilized by the processing arrangement with potentially no source code changes and combines operations to be performed simultaneously or parallelly that otherwise would be executed individually into a sequence of computation kernels. Beneficially, the implementation of the XLA scheme improves the speed and memory utilization capabilities due to elimination of intermediate storage buffers of the decoder of the encoder-decoder architecture and thereby improving the efficiency of the system. It will be appreciated that the accelerated linear algebra scheme is versatile in nature and various types of software (or ML) frameworks, models and/or libraries may be processed via the accelerated linear algebra scheme without any limitations in order to improve the efficiency of the system. Alternatively stated, the XLA schemes implemented via the decoder may be generated using different types of ML libraries and/or models such as, but not limited to, TensorFlow®, JAXR (Composable transformations of Python and NumPy programs), Julia® (The Julia language for scientific computing), PyTorch® (PyTorch framework), Nx® (Numerical computing library for the Elixir® programming language).

In one or more embodiments, the system further comprises one or more switches, provided via a user-interface, to allow a user to select a combination of one of the encoder, one of the decoder, one of the attention mechanism, one of the predefined parameters, and one of the textual identifier as the output. Typically, the system via provision of the user interface may allow the user to select various combinations of encoders, decoders, attention mechanisms and predefined parameters based on the requirement of the implementation to beneficially improve the accuracy and efficiency of the system. The term “switch” refers to input/output modules of the user interface configured to detect an input from the user and correspondingly provide an output based on the detected input. For example, the switch may be an on-off switch, configured to enable or disable one or more of the encoders or decoders of the encoder-decoder architecture, or enable or disable a particular attention mechanism associated with the selected decoder. The “user interface” may be a graphical user interface (GUI) or a command line interface (CLI) that may allow users to operate the one or more switches for selecting the required combination and/or number of the encoders and decoders, the associated attention mechanism and one or more predefined parameters used to process the input image of the structural formula of the chemical molecule to molecular translations as per the user requirements. Notably, the system is enabled with a modular encoder-decoder architecture for translation of the standardized images into textual identifiers that may be selectively enabled or disabled owing to the modularity (or modular code) of the computer program associated with the system.

The processing arrangement is further configured to recurrently process each of the features in the standardized image to predict corresponding unique token based on the associated unique tokens therewith to generate multiple possible sequences complementary to the textual identifier, and dynamically calculate a correctness probability for each of the generated multiple possible sequences based on a confidence of each prediction of corresponding unique tokens involved therein. The processing arrangement comprises the encoder-decoder architecture having multiple encoders and decoders associated with one or more machine learning (ML) models configured to recurrently and/or parallelly process each of the features in the standardized image i.e., features associated with each unique token, to generate an output as predicted token(s) based on the associated unique token(s) therewith in a faster and efficient manner. Notably, the recurrent processing refers to the processing of each token done sequentially i.e., step-by-step using the multiple encoders of the encoder-decoder architecture. The processing arrangement utilizes recurrent neural networks (RNN) to recurrently process each feature of the unique tokens to generate the predicted token(s) for generating the multiple possible sequences complementary to the textual identifier. Herein, the sequences refer to collection of predicted tokens and the permutations thereof. For example, the processing arrangement may receive an image and generate a score for each of a set of classes, with the score for a given class representing a correctness probability of the image containing an image of an object to belong to the given class. The RNN utilized herein may be composed of, e.g., a single level of linear or non-linear operations or may be a deep network, i.e., a ML model that is composed of multiple levels, one or more of which may be layers of non-linear operations. An example of a deep network is a neural network with one or more hidden layers. The RNNs are used to describe modelling methodology along with its parameters and hyperparameter settings. As used herein, the term “modelling methodology” refers to a machine learning technique, including supervised, unsupervised, and semi-supervised machine learning techniques. Non-limiting examples of model methodologies include support vector machine (SVM), neural networks (NN), Bayesian networks (BN), deep neural networks (DNN), deep belief networks (DBN), stochastic gradient descent (SGD), and random forest (RF). Beneficially, the recurrent processing of each of the features in the standardized image allows the RNN of the processing arrangement to obtain sequential characteristics and/or patterns to predict the next token for the textual identifier.

In one or more embodiments, the processing arrangement is configured to implement a beam search technique for the recurrent processing of each of the features in the standardized image of the structural formula for predicting corresponding unique token based on the associated unique tokens therewith to generate multiple possible sequences complementary to the textual identifier. The “beam search technique” refers to a heuristic search algorithm that examines the features (or embeddings) by selecting the encoder having the maximum correctness probability for the recurrent processing of each of the features in the standardized image. The beam search technique refers to a decoding mechanism or technique of the decoder configured to provide an improved prediction accuracy compared to greedy search algorithms for language generation (NLP) tasks. Herein, the decoder is configured to perform beam search with 1 channel i.e., width=1, wherein during beam search, the decoder selects N-tokens having highest conditional probabilities from the vocabulary (or ontology) at each step based on previously generated tokens (or history) i.e., the beam search with N channels or width=‘N’ for higher accuracy. The beamwidth bounds are set in order to efficiently utilize memory needed to complete the search while maintaining the accuracy and optimality of the textual identifiers generated via the processing arrangement. At each step, beam search provides a total of N×M number of probabilities, wherein ‘M’ refers to vocabulary length. Beneficially, the heuristic aspect of the beam search technique reduces the computational time and power cost associated with the selection of qualified nodes.

Moreover, the processing arrangement is further configured to dynamically calculate a correctness probability for each of the generated multiple possible sequences based on a confidence of each prediction of corresponding unique tokens involved therein to determine the accuracy of the predicted token. The term “confidence” as used herein refers to a confidence score, value, or range, associated with the accuracy of the predicted token. For example, the confidence may refer to a confidence interval of the predicted sequence in a range likely to contain the mean value of the dependent variable given specific values of the independent variables. The term “correctness probability” refers to a probability associated with the accuracy of the generated multiple possible sequence based on a confidence of each prediction. The processing arrangement, upon generation of the multiple possible sequences, is further configured to determine the accuracy of the predicted token by dynamic calculation of the correctness probability for each of the generated sequences to allow analysis of the performance of each layer of the encoder-decoder architecture and thereby allowing the system to potentially modify or remove the associated layer (such as, associated encoder or decoder) having lowest correctness probability. In an example, the processing arrangement is configured to rank the possible sequences based on their correctness probabilities and provide the top N-scoring at each step. Further, upon ranking, invalid molecular identifiers that do not correspond to any known molecules are removed for provision of the single best scoring valid prediction as the output i.e., the textual identifier.

The processing arrangement is further configured to select one of the multiple possible sequences with highest calculated correctness probability; and generate the textual identifier, as an output, for the image of the structural formula of the chemical molecule based on the selected one of the multiple possible sequences. That is, based on the correctness probability of each of the multiple possible sequences complimentary to the textual identifier, the sequence with the highest correctness probability is selected as the output i.e., the generated textual identifier selected from the multiple possible sequences. Herein, as discussed, the textual identifier, as the output, is in the form of one or more of: an International Chemical Identifier (InChI) textual identifier, Simplified molecular-input line-entry (SMILE) textual identifier, Javascript object notation (JSON) files with each sublayer of the chemical notation as a separate field.

In one or more embodiments, the processing arrangement is further configured to implement a dataset of textual identifiers of known chemical molecules and compare the generated textual identifier to the textual identifiers of the known chemical molecules. Further, based on the comparison, the processing arrangement is configured to determine if there is no match of the generated textual identifier to any one of the textual identifiers of the known chemical molecules and thereby simultaneously select one of the multiple possible sequences with next highest calculated correctness probability.

In a second aspect, the present disclosure provides a computer readable storage medium having computer executable instruction that when executed by a computer system, causes the computer system to execute a method for translating an image of a structural formula of a chemical molecule into a textual identifier therefor utilizing unique tokens generated for each of known entities in chemical molecules.

The method comprises pre-processing the image of the structural formula of the chemical molecule to generate a standardized image of the structural formula based on predefined parameters, processing the standardized image of the structural formula using an encoder-decoder architecture, wherein an encoder is implemented to generate embeddings for features in the standardized image of the structural formula and a decoder is implemented to utilize the generated embeddings along with an attention mechanism to associate each of the features in the standardized image of the structural formula to one of the unique tokens, recurrently processing each of the features in the standardized image of the structural formula for predicting corresponding unique token based on the associated unique tokens therewith to generate multiple possible sequences complementary to the textual identifier, and dynamically calculating a correctness probability for each of the generated multiple possible sequences based on a confidence of each prediction of corresponding unique tokens involved therein, selecting one of the multiple possible sequences with highest calculated correctness probability and generating the textual identifier, as an output, for the image of the structural formula of the chemical molecule based on the selected one of the multiple possible sequences.

In one or more embodiments, the method further comprises implementing a dataset of textual identifiers of known chemical molecules, comparing the generated textual identifier to the textual identifiers of the known chemical molecules, determining if there is no match of the generated textual identifier to any one of the textual identifiers of the known chemical molecules and simultaneously (i.e., in a single step) selecting one of the multiple possible sequences with next highest calculated correctness probability.

In one or more embodiments, the processing (preprocessing) the standardized image of the structural formula using the encoder-decoder architecture is a tensor workflow as a TensorFlow TFRecord.

In one or more embodiments, the encoder is configured to implement a mixed precision accuracy scheme to the embeddings for the features in the TensorFlow TFRecord for improving the computational efficiency of the processing arrangement while maintaining a high accuracy.

In one or more embodiments, the method further comprises implementing a beam search technique for the recurrent processing of each of the features in the standardized image of the structural formula for predicting corresponding unique token based on the associated unique tokens therewith to generate multiple possible sequences complementary to the textual identifier.

In one or more embodiments, the encoder implements one or more of: an EfficientNet encoder, an EfficientNetV2 encoder, a Vision Transformer (ViT) encoder.

In one or more embodiments, the decoder implements one or more of: a Recurrent Neural Network (RNN) with a Gated Recurrent Unit (GRU) decoder, a RNN with a Long Short-Term Memory (LSTM) decoder, a Transformer with self-attention decoder.

In one or more embodiments, the attention mechanism implements one or more of: Bahdanau Attention, Transformer self-attention.

In one or more embodiments, the predefined parameters comprise one or more of: a crop parameter for pre-processing the image of the structural formula of the chemical molecule, an aspect ratio parameter for pre-processing the image of the structural formula of the chemical molecule, a color inversion parameter for pre-processing the image of the structural formula of the chemical molecule.

In one or more embodiments, the textual identifier, as the output, is one or more of: an International Chemical Identifier (InChI) textual identifier, Simplified molecular-input line-entry (SMILE) textual identifier, JavaScript object notation (JSON) files with each sublayer of the chemical notation as a separate field.

In one or more embodiments, the method further comprises providing one or more switches, provided via a user-interface, to allow a user to select a combination of one of the encoder, one of the decoder, one of the attention mechanism, one of the predefined parameters, and one of the textual identifier as the output.

In a third aspect, the present disclosure provides a computer program comprising computer executable program code, when executed the computer executable program code controls a computer system to perform the method. Notably, the computed program provided in the present disclosure has a modular program code that enables the method and system to selectively utilize the encoder-decoder architecture of the processing arrangement for translating the image of a structural formula of a chemical molecule into the textual identifier therefor.

### DETAILED DESCRIPTION OF THE DRAWINGS

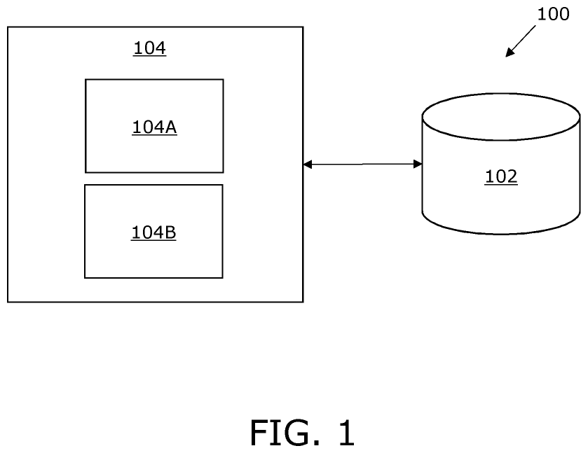

Referring to FIG. 1, illustrated is a block diagram of a system 100 for translating an image of a structural formula of a chemical molecule into a textual identifier therefor, in accordance with an embodiment of the present disclosure. As shown, the system 100 comprises a database 102 configured to store unique tokens defined for each of known entities in chemical molecules and a processing arrangement 104. The processing arrangement is configured to pre-process the image of the structural formula of the chemical molecule to generate a standardized image of the structural formula based on predefined parameters. Further, the processing arrangement 104 is configured to process the standardized image of the structural formula using an encoder-decoder architecture, wherein an encoder 104A is implemented to generate embeddings for features in the standardized image of the structural formula and a decoder 104B is implemented to utilize the generated embeddings along with an attention mechanism to associate each of the features in the standardized image of the structural formula to one of the unique tokens. Further, the processing arrangement 104 is configured to recurrently process each of the features in the standardized image of the structural formula for predicting corresponding unique token based on the associated unique tokens therewith to generate multiple possible sequences complementary to the textual identifier, and dynamically calculate a correctness probability for each of the generated multiple possible sequences based on a confidence of each prediction of corresponding unique tokens involved therein, select one of the multiple possible sequences with highest calculated correctness probability, and generate the textual identifier, as an output, for the image of the structural formula of the chemical molecule based on the selected one of the multiple possible sequences.

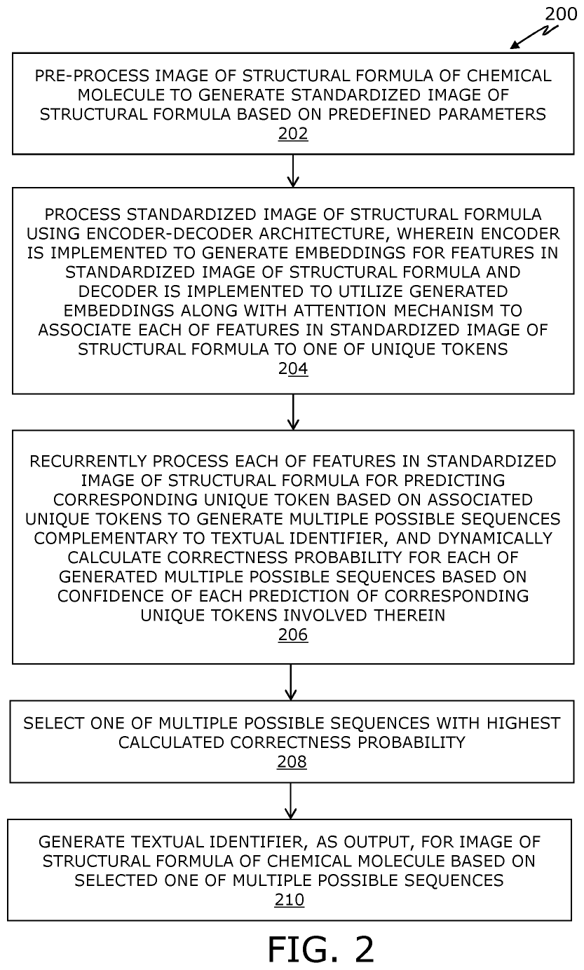

Referring to FIG. 2, illustrated is a flowchart listing steps involved in a method 200 for translating an image of a structural formula of a chemical molecule into a textual identifier therefor, in accordance with an embodiment of the present disclosure. At step 202, the method 100 comprises pre-processing the image of the structural formula of the chemical molecule to generate a standardized image of the structural formula based on predefined parameters. At step 104, the method 100 further comprises processing the standardized image of the structural formula using an encoder-decoder architecture, wherein an encoder is implemented to generate embeddings for features in the standardized image of the structural formula and a decoder is implemented to utilize the generated embeddings along with an attention mechanism to associate each of the features in the standardized image of the structural formula to one of the unique tokens. At step 106, the method further comprises recurrently processing each of the features in the standardized image of the structural formula for predicting corresponding unique token based on the associated unique tokens therewith to generate multiple possible sequences complementary to the textual identifier, and dynamically calculating a correctness probability for each of the generated multiple possible sequences based on a confidence of each prediction of corresponding unique tokens involved therein. At step 108, the method further comprises selecting one of the multiple possible sequences with highest calculated correctness probability and, at step 110, the method further comprises generating the textual identifier, as an output, for the image of the structural formula of the chemical molecule based on the selected one of the multiple possible sequences. The aforementioned steps are only illustrative and other alternatives can also be provided where one or more steps are added, one or more steps are removed, or one or more steps are provided in a different sequence without departing from the scope of the claims herein.

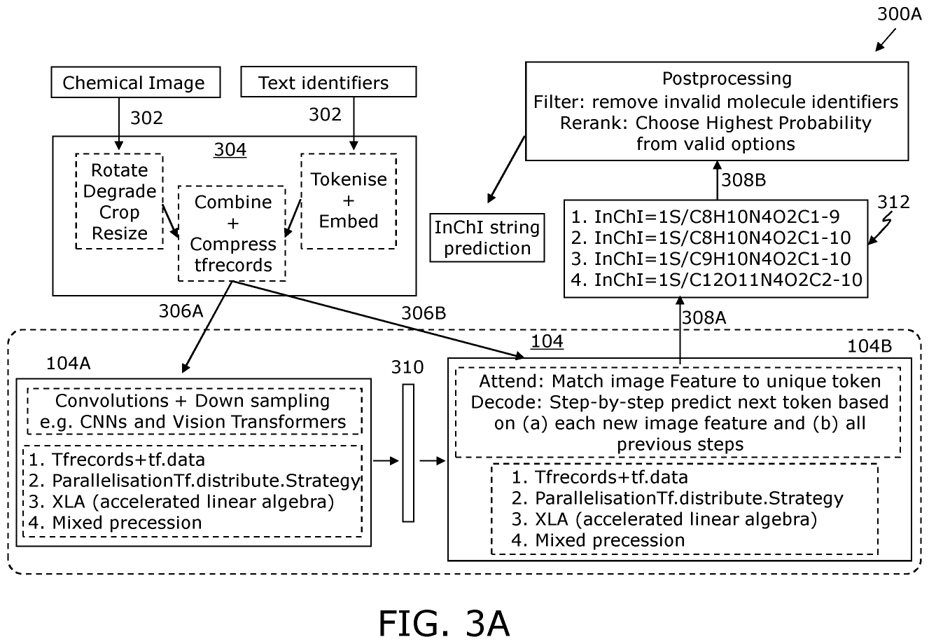

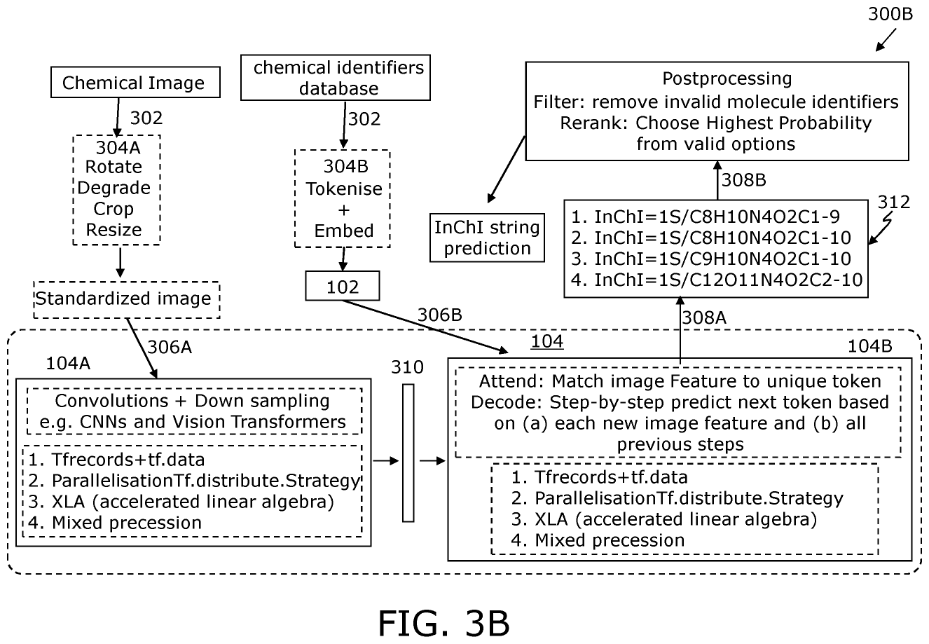

Referring to FIG. 3A, illustrated is a schematic block diagram depicting a pipeline 300A involved in the method 200 for translating an image of a structural formula of a chemical molecule into a textual identifier therefor, in accordance with an embodiment of the present disclosure. Herein, the training phase of the machine learning model is depicted. As shown, at block 302, an image of the structural formula of the chemical molecule (or the chemical image) and the corresponding textual identifier are received via the processing arrangement 104. Further, at block 304, the input data i.e., the chemical image and the textual identifier are pre-processed via the processing arrangement 104 based on one or more parameters to generate a standardized image (e.g., compressed TFrecords) to be fed to an encoder 104A of the encoder-decoder architecture (or ML model). Herein, the chemical image is modified using at least one of rotation, degradation, cropping, resizing of the input chemical image; whereas, the textual identifiers are tokenized to generate unique tokens to be embedded thereafter, to be combined and compressed to generate the standardized image for further processing. Further, at block 306, the pipeline 300A comprises two sub-steps i.e., at sub-step 306A, the encoder 104A is configured to generate embeddings 310 (i.e., encoded feature vector) for the standardized image to be fed into a decoder 104B by performing down sampling and convolution such as, using CNN encoder or VIT encoders. Furthermore, at sub-step 306B, the decoder 104B is configured to utilize the generated embeddings along with an attention mechanism to extract or associate features of the standardized image to the unique tokens in a recurrent manner. Furthermore, at block 308, the pipeline 300 comprises two sub-steps i.e., at sub-step 308A, the processing arrangement 104 is configured to recurrently process each of the features, as obtained from the block 306, in the standardized image of the structural formula for predicting corresponding unique token based on the associated unique tokens therewith to generate multiple possible sequences 312; and at sub-step 308B, the processing arrangement 104 is configured to calculate a correctness probability for each of the generated multiple possible sequences based on a confidence of each prediction of corresponding unique tokens involved therein. Lastly, at step 308B, the processing arrangement 104 is configured to perform one or more post-processing techniques for selecting one of the possible sequences with highest calculated correctness probability and generate the textual identifier, as an output, for the image of the structural formula of the chemical molecule based on the selected one of the multiple possible sequences.

Referring to FIG. 3B, illustrated is a schematic block diagram depicting a pipeline 300B involved in the method 200 for translating an image of a structural formula of a chemical molecule into a textual identifier therefor, in accordance with another embodiment of the present disclosure. Herein, the inference phase of the machine learning model is depicted. As shown, at block 302A and 302B, an image of the structural formula of the chemical molecule (or the chemical image) and a (preconfigured) chemical identifier database is received via the processing arrangement 104. Further, at block 304A, the input chemical image is pre-processed via the processing arrangement 104 based on one or more parameters to generate a standardized image (e.g., compressed TFrecords) to be fed to an encoder 104A of the encoder-decoder architecture (or ML model). Herein, the chemical image is modified using at least one of rotation, degradation, cropping, resizing of the input chemical image; whereas, the textual identifiers are tokenized to generate unique tokens to be embedded thereafter, to be combined and compressed to generate the standardized image for further processing. Moreover, at block 304B, the input chemical identifiers database 302 is utilized to tokenize and embed the known entities of the input chemical image in the database 102 for matching and/or classification thereof. Further, at block 306, the pipeline 300B comprises two sub-steps i.e., at sub-step 306A, the encoder 104A is configured to generate embeddings 310 (i.e., encoded feature vector) for the standardized image to be fed into a decoder 104B by performing down sampling and convolution such as, using CNN encoder or ViT encoders. Furthermore, at sub-step 306B, the decoder 104B is configured to utilize the generated embeddings along with an attention mechanism to extract or associate features of the standardized image to the unique tokens in a recurrent manner. Furthermore, at block 308, the pipeline 300B comprises two sub-steps i.e., at sub-step 308A, the processing arrangement 104 is configured to recurrently process each of the features, as obtained from the block 306A, in the standardized image of the structural formula for predicting corresponding unique token based on the associated unique tokens therewith to generate multiple possible sequences 312; and at sub-step 308B, the processing arrangement 104 is configured to calculate a correctness probability for each of the generated multiple possible sequences based on a confidence of each prediction of corresponding unique tokens involved therein. Lastly, at step 308B, the processing arrangement 104 is configured to perform one or more post-processing techniques for selecting one of the possible sequences with highest calculated correctness probability and generate the textual identifier, as an output, for the image of the structural formula of the chemical molecule based on the selected one of the multiple possible sequences.

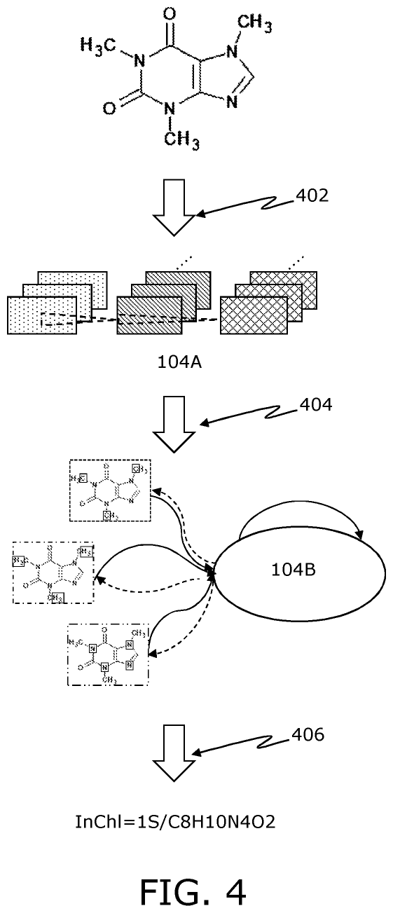

Referring to FIG. 4, illustrated is an exemplary flowchart of steps for translating an exemplary image into a textual description therefor as involved in the method 200, in accordance with an embodiment of the present disclosure. Herein, as shown, the input image depicting structural formula for caffeine is received via the processing arrangement 104 (for example, TPU for faster processing) to translate the input chemical image into textual identifiers. At step 402, the encoder 104A of the processing arrangement 104 is configured to generate convolutional layers of the input image, wherein the generation of the convolution layers may be performed by extracting one or more features from the input image and thereby associating features to each of the one or more unique tokens to generate the standardized image. The features may be extracted using convolution feature extraction on each of the one or more unique tokens. At step 404, the processing arrangement 104 may be configured to utilize the extracted features associated with the unique tokens for generating a multi-dimensional feature map associated with the one or more features. In an exemplary scenario, wherein an input image is a bird, the processing arrangement 104 may generate a 14×14 convolution feature matrix to be further processed for generating the standardized image. Further, the encoder 104A may compress or condense the standardized image to enhance and/or improve the extracted features of the standardized image through the generated pooling layers configured for down sampling to be further processed via the decoder 104B. Furthermore, the encoder 104A may be configured to convert, through down sampling via the pooling layers, the embeddings into one or more flattened layers (or vectors) that are suitable for efficient processing via the decoder 104B of the encoder-decoder architecture of the processing arrangement 104. Furthermore, at step 404, the processing arrangement 104 is configured to implement an attention-based LSTM model configured to automatically learn description or analysis of content and/or context of the input chemical image via the generated embeddings. The model is trained in a deterministic and recurrent t manner using standard backpropagation techniques. Additionally, the generated multi-dimensional feature matrix may be visualized by the model to be trained to automatically locate and processing salient features of the standardized image for generating the corresponding textual identifier using word by word or token by token generation. Optionally, the processing arrangement 104 may validate the use of attention with state-of-the-art performance on various benchmark datasets such as, but not limited to, Flickr30k and MS COCO. Further, at step 406, the decoder 104B of the processing arrangement 104 is further configured to recurrently process each of the features in the standardized image of structural formula for predicting corresponding unique token based on the associated unique tokens therewith to generate multiple possible sequences complementary to the textual identifier and dynamically calculate a correctness probability for each of the generated multiple possible sequences based on a confidence of each prediction of corresponding unique tokens involved therein, select one of the multiple possible sequences with highest calculated correctness probability and generate the textual identifier, as an output, for the image of the structural formula of the chemical molecule based on the selected one of the multiple possible sequences. The aforementioned steps are only illustrative and other alternatives can also be provided where one or more steps are added, one or more steps are removed, or one or more steps are provided in a different sequence without departing from the scope of the claims herein.

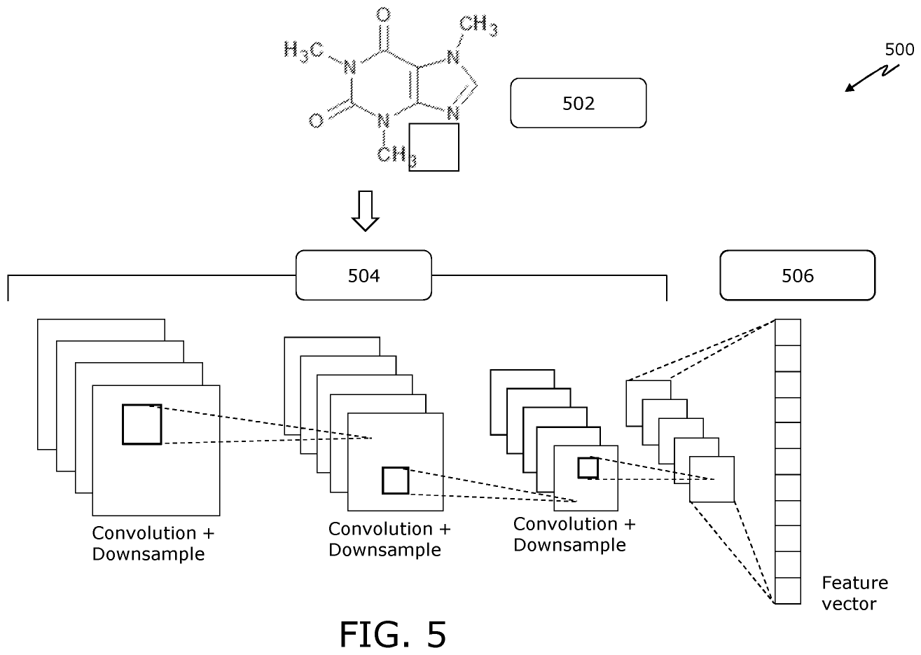

Referring to FIG. 5, illustrated is a simplified pipeline 500 of an embedding generation process implemented via the method 200 for translating an image of a structural formula of a chemical molecule into a feature vector for generation of textual identifier therefor, in accordance with one or more embodiments of the present disclosure. As shown, the encoder 104A may be configured to generate the one or more embeddings by performing one or more processing techniques to filter a pre-processed image 502 (or the standardized image) for detecting elements in the preprocessed chemical image 502 and extracting specific features (or parameters) therein via implementation of convolutional layers for enabling detection and extraction of required features of each of the unique tokens in the filtered standardized image 502. Further, the encoder 104A may be configured to convert the extracted features into a feature map 504 that may be passed through an activation function i.e., the ReLU activation function, wherein weights (or kernels) may be assigned via the system 100 to each unique token (or parameter) of the standardized image 502 to define the interconnection between the convolutional layers and thereby extract the features from the standardized image 502. Further, the encoder 104A may compress or condense the standardized image 502 to enhance and/or improve the extracted features of the standardized image 502 to generate pooling layers to be further processed via the decoder 104B. Furthermore, the encoder 104A may be configured to convert the pooling layers into the embeddings i.e., one or more flattened layers (or vectors) that are suitable for efficient processing via the decoder 104B of the encoder-decoder architecture of the processing arrangement 104 and thereby enabling generation of accurate low dimensional feature vectors 506 in the form of flattened layers associated with the input image of the chemical molecule in an efficient manner to be further utilized for the generation of the textual identifiers.

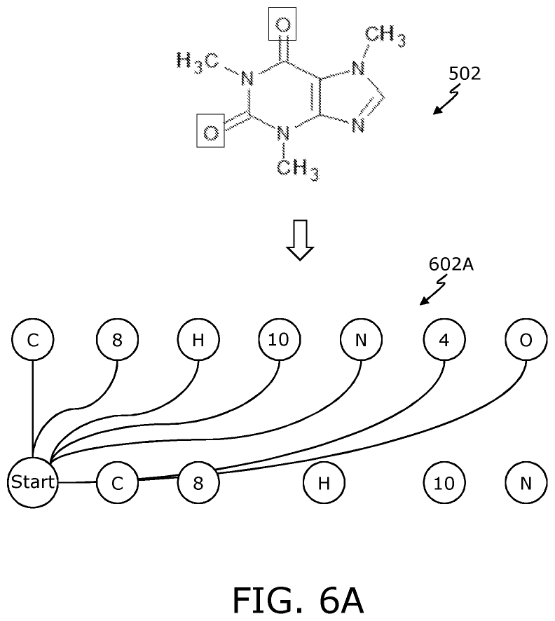

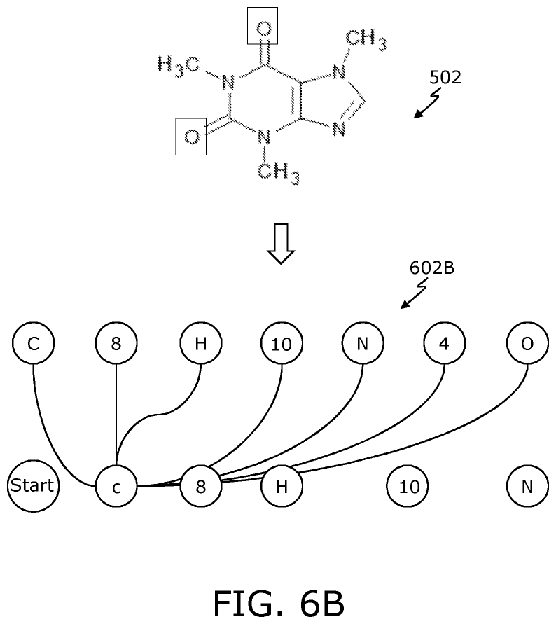

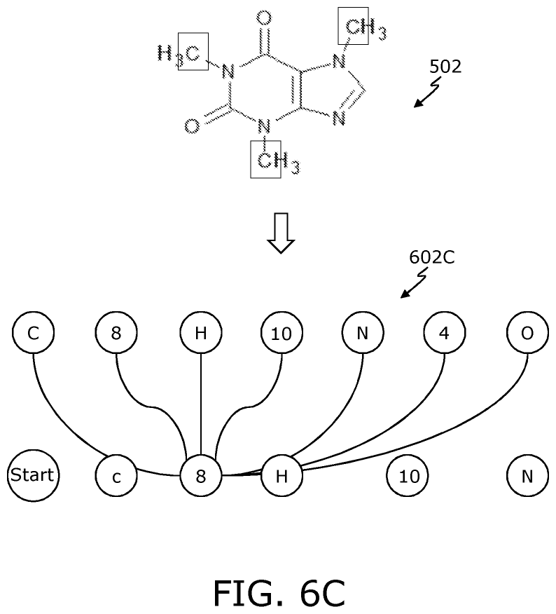

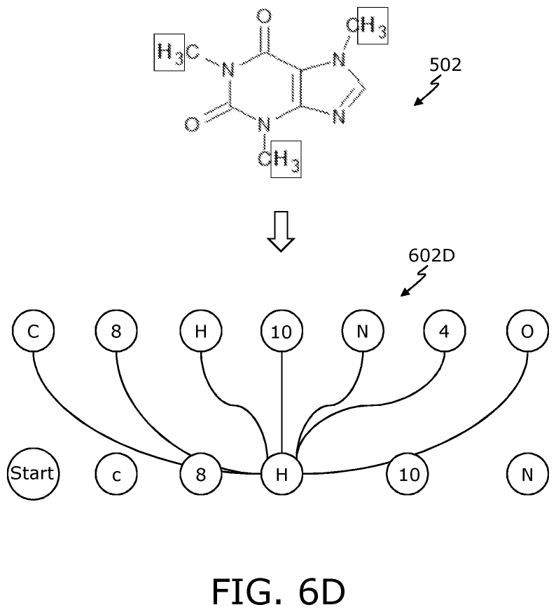

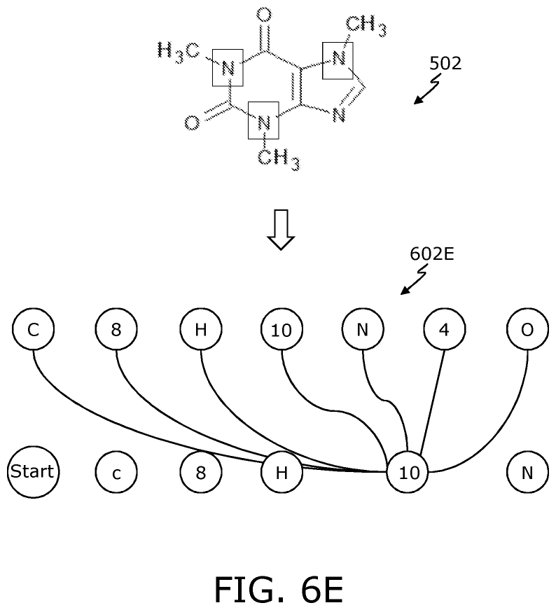

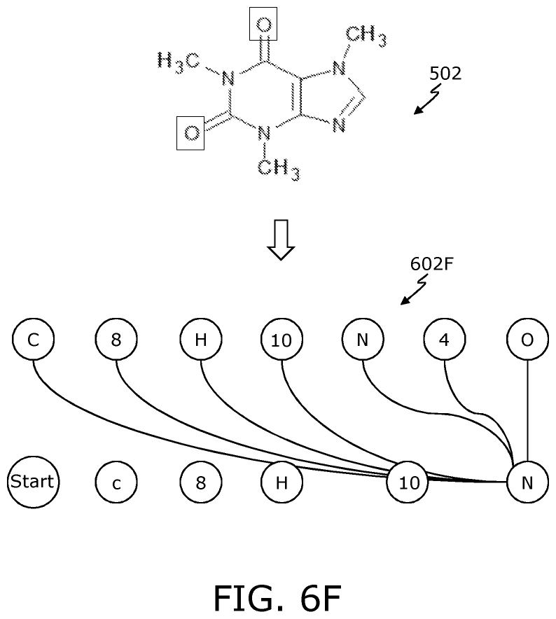

Referring to FIGS. 6A to 6F, illustrated are exemplary depictions of the generation of multiple possible sequences complementary to the textual identifier via the decoder 104B of the processing arrangement 104, in accordance with one or more embodiments of the present disclosure. As shown, the input chemical image 502 depicts the structural formula of caffeine, wherein upon preprocessing of the input chemical image into the standardized image and generation of embeddings via the encoder 104A for features in the standardized image, the decoder 104B of the processing arrangement 104 is configured to recurrently process each of the features, for example, each of the elements in the chemical molecule i.e., Carbon, Nitrogen, Hydrogen, and Oxygen and the number of each element in the chemical molecule (i.e., caffeine) in the standardized image of structural formula for predicting corresponding unique token based on the associated unique tokens therewith to generate multiple possible sequences 602A to 602F (similar to multiple possible sequence 312) complementary to the textual identifier to generate the textual identifier, as an output, for the image 502 of the structural formula of the chemical molecule based on the selected one of the multiple possible sequences 602A to 602F as shown in each of the FIGS. 6A to 6F, respectively. Herein, in one embodiment, the decoder may be a GRU implementing Bahdanau attention mechanism with variable sizes; in another embodiment, the decoder may be a LSTM implementing Badhanau attention mechanism with variable sizes; in yet another embodiment, the decoder may be a transformer implementing self-attention mechanism, based on the requirements of the implementation.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

What is claimed is:

1. A system for translating an image of a structural formula of a chemical molecule into a textual identifier therefor, the system comprising:
a database configured to store unique tokens defined for each of known entities in chemical molecules; and
a processing arrangement configured to:
pre-process the image of the structural formula of the chemical molecule to generate a standardized image of the structural formula based on predefined parameters;
process the standardized image of the structural formula using an encoder-decoder architecture, wherein an encoder is implemented to generate embeddings for features in the standardized image of the structural formula and a decoder is implemented to utilize the generated embeddings along with an attention mechanism to associate each of the features in the standardized image of the structural formula to one of the unique tokens;
recurrently process each of the features in the standardized image of the structural formula for predicting corresponding unique token based on the associated unique tokens therewith to generate multiple possible sequences complementary to the textual identifier, and dynamically calculate a correctness probability for each of the generated multiple possible sequences based on a confidence of each prediction of corresponding unique tokens involved therein;
select one of the multiple possible sequences with highest calculated correctness probability; and
generate the textual identifier, as an output, for the image of the structural formula of the chemical molecule based on the selected one of the multiple possible sequences.

2. The system according to claim 1, wherein the processing arrangement is further configured to:
implement a dataset of textual identifiers of known chemical molecules;
compare the generated textual identifier to the textual identifiers of the known chemical molecules;
determine if there is no match of the generated textual identifier to any one of the textual identifiers of the known chemical molecules; and
select one of the multiple possible sequences with next highest calculated correctness probability.

3. The system according to claim 1, wherein the processing arrangement implements a tensor workflow for the encoder-decoder architecture for processing the standardized image of the structural formula as a TensorFlow TFRecord.

4. The system according to claim 3, wherein the encoder, in the processing arrangement, is configured to implement a mixed precision accuracy scheme to the embeddings for the features in the TensorFlow TFRecord.

5. The system according to claim 1, wherein the processing arrangement is configured to implement a beam search technique for the recurrent processing of each of the features in the standardized image of the structural formula for predicting corresponding unique token based on the associated unique tokens therewith to generate multiple possible sequences complementary to the textual identifier.

6. The system according to claim 1, wherein:
the encoder implements one or more of: an EfficientNet encoder, an EfficientNetV2 encoder, a Vision Transformer (ViT) encoder,
the decoder implements one or more of: a Recurrent Neural Network (RNN) with a Gated Recurrent Unit (GRU) decoder, a RNN with a Long Short-Term Memory (LSTM) decoder, a Transformer with self-attention decoder,
the attention mechanism implements one or more of: Bahdanau Attention, Transformer self-attention,
the predefined parameters comprise one or more of: a crop parameter for pre-processing the image of the structural formula of the chemical molecule, an aspect ratio parameter for pre-processing the image of the structural formula of the chemical molecule, a color inversion parameter for pre-processing the image of the structural formula of the chemical molecule, and
the textual identifier, as the output, is one or more of: an International Chemical Identifier (InChI) textual identifier, Simplified molecular-input line-entry (SMILE) textual identifier, JSON files with each sublayer of the chemical notation as a separate field.

7. The system according to claim 6 further comprising one or more switches, provided via a user-interface, to allow a user to select a combination of one of the encoder, one of the decoder, one of the attention mechanism, one of the predefined parameters, and one of the textual identifier as the output.

8. A computer readable storage medium having computer executable instruction that when executed by a computer system, causes the computer system to execute a method for translating an image of a structural formula of a chemical molecule into a textual identifier therefor utilizing unique tokens generated for each of known entities in chemical molecules, the method comprising:
pre-processing the image of the structural formula of the chemical molecule to generate a standardized image of the structural formula based on predefined parameters;
processing the standardized image of the structural formula using an encoder-decoder architecture, wherein an encoder is implemented to generate embeddings for features in the standardized image of the structural formula and a decoder is implemented to utilize the generated embeddings along with an attention mechanism to associate each of the features in the standardized image of the structural formula to one of the unique tokens;
recurrently processing each of the features in the standardized image of the structural formula for predicting corresponding unique token based on the associated unique tokens therewith to generate multiple possible sequences complementary to the textual identifier, and dynamically calculating a correctness probability for each of the generated multiple possible sequences based on a confidence of each prediction of corresponding unique tokens involved therein;
selecting one of the multiple possible sequences with highest calculated correctness probability; and
generating the textual identifier, as an output, for the image of the structural formula of the chemical molecule based on the selected one of the multiple possible sequences.

9. The method according to claim 8 further comprising:
implementing a dataset of textual identifiers of known chemical molecules;
comparing the generated textual identifier to the textual identifiers of the known chemical molecules;
determining if there is no match of the generated textual identifier to any one of the textual identifiers of the known chemical molecules; and
selecting one of the multiple possible sequences with next highest calculated correctness probability.

10. The method according to claim 8, wherein the processing the standardized image of the structural formula using the encoder-decoder architecture is a tensor workflow as a TensorFlow TFRecord.

11. The method according to claim 10, wherein the encoder is configured to implement a mixed precision accuracy scheme to the embeddings for the features in the TensorFlow TFRecord.

12. The method according to claim 8 further comprising implementing a beam search technique for the recurrent processing of each of the features in the standardized image of the structural formula for predicting corresponding unique token based on the associated unique tokens therewith to generate multiple possible sequences complementary to the textual identifier.

13. The method according to claim 8, wherein:
the encoder implements one or more of: an EfficientNet encoder, an EfficientNetV2 encoder, a Vision Transformer (ViT) encoder,
the decoder implements one or more of: a Recurrent Neural Network (RNN) with a Gated Recurrent Unit (GRU) decoder, a RNN with a Long Short-Term Memory (LSTM) decoder, a Transformer with self-attention decoder,
the attention mechanism implements one or more of: Bahdanau Attention, Transformer self-attention,
the predefined parameters comprise one or more of: a crop parameter for pre-processing the image of the structural formula of the chemical molecule, an aspect ratio parameter for pre-processing the image of the structural formula of the chemical molecule, a color inversion parameter for pre-processing the image of the structural formula of the chemical molecule, and
the textual identifier, as the output, is one or more of: an International Chemical Identifier (InChI) textual identifier, Simplified molecular-input line-entry (SMILE) textual identifier, JSON files with each sublayer of the chemical notation as a separate field.

14. The method according to claim 13 further comprising providing one or more switches, provided via a user-interface, to allow a user to select a combination of one of the encoder, one of the decoder, one of the attention mechanism, one of the predefined parameters, and one of the textual identifier as the output.

15. A computer program comprising computer executable program code, when executed the computer executable program code controls a computer system to perform the method according to claim 8.
